(19) United States
(12) Patent Application Publication
(10) Pub. No.: US 2019/0274623 A1
(43) Pub. Date: Sep. 12, 2019

(54) DEVICE AND SYSTEM FOR MONITORING PHYSIOLOGICAL SIGNALS FROM A FINGER

(71) Applicant: MEDASENSE BIOMETRICS LTD, Ramat Gen (IL)

(72) Inventors: Galit ZUCKERMAN-STARK, Tel Aviv (IL); Adi COSTA, Kfar Saba (IL); Shimon HAYUN, Rosh Haayin (IL); Refael Gil KOBY, Kochav Yair (IL); Naum MUCHNIK, Yokneam-Ilit (IL); Avi KAYTON, Tel Aviv (IL)

(21) Appl. No.: 16/343,086

(22) PCT Filed: Oct. 19, 2017
(86) PCT No.: PCT/IL2017/051154

§ 371 Date: Apr. 18, 2019

Related U.S. Application Data
(60) Provisional application No. 62/410,946, filed on Oct. 21, 2016.

(51) Int. Cl. A61B 5/00; A61B 5/0205; A61B 5/053

(52) U.S. Cl. CPC A61B 5/4824; A61B 5/02055; A61B 5/6826; A61B 5/6831; A61B 5/02427; A61B 5/0533; A61B 5/6833; A61B 2562/0271; A61B 5/0008

(57) ABSTRACT

A finger probe for acquiring physiological signals of a subject, the finger probe including a housing and a body portion, the body portion configured to hold therein the housing in a liquid tight manner, wherein the body portion includes a strap configured to secure the finger probe to the subject's finger, a longitudinal groove configured to accommodate the subject's finger, and a hood configured to cover the distal end of the subject's finger from an opposite side thereof, and wherein the housing includes a Printed Circuit Board, a PPG sensor and at least one additional sensor.

### TECHNICAL FIELD

The present disclosure generally relates to the field of physiological signals monitoring, specifically to finger probes including sensors for monitoring physiological signals used for estimation of pain.

### BACKGROUND

The sensation of pain is an extremely complex interaction of biological, cognitive, behavioral, cultural, and environmental factors. Yet the reaction of the body to an injury or noxious stimulus, e.g., an acute pain, is first and foremost a physiological response due to activation of the autonomic neural and hormonal pathways by a nociceptive stimulus. Nociception refers to the detection, transduction, and transmission of noxious stimuli that elicits an autonomic response even in an unconscious subject. Over the years, multiple studies have investigated nociception-related changes in different physiological parameters as the basis for objective assessment of the level of nociception during surgery.

The skin conductance response is the phenomenon that the skin momentarily becomes a better conductor of electricity when perspiration increases. A subject who has been exposed to a physiologically arousing situation will therefore display a sudden drop in resistance between two areas of the skin. A correlation between skin conductance and pain has also been demonstrated, since skin conductance is elevated in response to nociception. Determination of skin conductance is typically based on measurements obtained from an active electrode configured to induce an electrical signal such as an electrical current, and an inactive electrode configured to collect the electrical signal.

The photo-plethysmographic waveform can provide information about parameters such as heart rate (HR), heart rate variability (HRV) and photo-plethysmographic amplitude (PPGA). These parameters are known as indicators of the autonomic function and nociceptive response.

While the above parameters may have a good correlation with the subject's pain level, confounders often cause a false detection. Integration of additional sensors, such as an accelerometer, thermometer and others, can provide the ability to reduce misdetection and increase the specificity of the subject's pain level.

### SUMMARY

Aspects of the disclosure, in some embodiments thereof, relate to a fingerprobe device including sensors capable of detecting pain.

A common problem when monitoring pain is the tradeoff between the ability to detect pain with high sensitivity and the aim of avoiding false positive readings.

Advantageously, the device disclosed herein is sized and shaped to include at least two sensors and to enable reliable monitoring of the subject's pain level from a single finger, while minimizing the number of false positive readings. The device is a stand-alone data acquisition and recorder of physiological signals from which, for example, a subject's pain level can be determined.

According to some embodiments, there is provided a finger probe for monitoring pain of a subject, the finger probe including a housing and a body portion configured to hold therein the housing. According to some embodiments, the body portion has a strap configured to secure the finger probe to the subject's finger, a first longitudinal groove configured to accommodate the subject's finger, and a hood configured to cover the distal end of the subject's finger from an opposite side thereof. According to some embodiments, the housing includes a Printed Circuit Board (PCB), a PPG sensor, and at least one additional sensor.

According to some embodiments, the strap includes a plurality of apertures configured to prevent heating of the subject's finger, when attached. According to some embodiments, the strap further includes a plurality of bulges configured to generate friction and prevent movement of the subject's finger once secured.

According to some embodiments, the body portion is made of a material having a first shore and the strap from a material having a second shore. According to some embodiments, the second shore is lower than the first shore.

According to some embodiments, the finger probe further includes two lateral slits configured to slidingly receive and secure wings of an electrode array.

According to some embodiments, the body portion includes a longitudinal aperture exposing at least an on/off button positioned on the housing.

According to some embodiments, the hood includes a second longitudinal groove. According to some embodiments, the first and second longitudinal grooves form a tunnel-like compartment configured to accommodate the subject's finger and to prevent penetration of ambient light.

According to some embodiments, the PPG sensor is positioned at a distal end of the housing underneath the tunnel-like compartment.

According to some embodiments, the body portion includes a PPG aperture within the tunnel-like compartment and above the PPG sensor, the PPG aperture configured to allow light transmitted by the PPG sensor to reach the subject's finger. According to some embodiments, the PPG aperture may be covered by a transparent cover allowing light transmitted by the PPG sensor to pass there through, while preventing penetration of liquids into the housing.

According to some embodiments, the at least one additional sensor includes a thermistor configured to monitor the skin temperature on the subject's finger. According to some embodiments, the thermistor is positioned at a distal end of the housing underneath the tunnel-like compartment.

According to some embodiments, the body portion further includes a thermistor aperture configured to allow contact between the thermistor and the subject's finger.

According to some embodiments, the body portion further includes a stopper positioned within the tunnel-like compartment. According to some embodiments, the stopper is configured to prevent forward movement of the subject's finger.

According to some embodiments, the at least one additional sensor further includes an accelerometer.

According to some embodiments, the PCB is electrically connected to the PPG, sensor, to the at least one additional sensor and to a rechargeable battery. According to some embodiments, the PCB is configured for wireless communication with a remote monitor and/or computer.

According to some embodiments, there is provided an electrode array configured to monitor galvanic skin resistance (GSR) on a subject's finger, the electrode array including an electrically conductive flexible portion mounting GSR electrodes and an interphase portion. According to some embodiments, the interphase portion is shaped to form at least two compartments configured to enable contact between the subject's finger and the GSR electrodes. According to some embodiments, the electrode array is shaped with lateral wings configured to secure the electrode array to a finger probe (such as the finger probe disclosed herein).

According to some embodiments, the at least two compartments include a conductive biocompatible gel capable of creating and/or enhancing galvanic contact between the subject's finger and the GSR electrodes.

According to some embodiments, the interphase portion (except the wings) includes (or is covered by) an adhesive on the side thereof configured to face the subject's finger. According to some embodiments, the adhesive may be covered by a removable cover configured to be removed prior to use.

According to some embodiments, a proximal end of the electrically conductive flexible portion includes an electrical connector configured to electrically connect the electrode array to a PCB of a finger probe (such as the PCB of the finger probe disclosed herein).

According to some embodiments, there is provided a pain monitoring system including the finger probe disclosed herein and the electrode array disclosed herein.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure. For the sake of clarity, some objects depicted in the figures are not to scale.

FIG. 1A shows a rear perspective view of a finger probe device for monitoring pain, including a body portion and a housing, according to some embodiments;

FIG. 1B shows a front perspective view of a finger probe device for monitoring pain, including a body portion and a housing, according to some embodiments;

FIG. 1C shows a front perspective view of a finger probe device for monitoring pain, including a body portion holding an electrode array and a housing, according to some embodiments;

FIG. 1D shows a cross sectional view of a finger probe device for monitoring pain, according to some embodiments.

FIG. 2 shows a perspective view of a finger probe housing, according to some embodiments;

FIG. 3 shows a perspective view of an uncovered housing (PCB circuit) connected to an electrode array, according to some embodiments;

FIG. 4 shows a perspective view of an electrode array, according to some embodiments;

FIG. 5 schematically illustrates a block diagram of the pain monitoring system, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to some embodiments, there is provided a pain monitoring device including a housing and a body portion surrounding the housing.

According to some embodiments, the housing may be made of a rigid material, such as a firm plastic. The housing includes a scaffold configured to hold a Printed Circuit Board (PCB), serving as a system on chip for acquiring multiple physiological signals. On the PCB is connected a photoplethysmograpgh (referred to herein as a PPG sensor) that includes a photodiode, and optionally one or more additional sensors such as a thermistor and/or an accelerometer. Other non-limiting examples of suitable sensors include a DCS (diffused correlation spectroscopy) sensor, an acoustics sensor, a bio-impedance sensor, a piezoelectric sensor, or any other suitable sensor of physiological parameters. Each possibility is a separate embodiment.

According to some embodiments, the thermistor may be configured to measure and/or determine the subject's skin temperature and changes therein. According to some embodiments, there may be more than one thermistor in multiple locations of the probe. That is, according to some embodiments, the set of thermistors may include at least one thermistor on a first side configured to contact the subject's finger and to measure the temperature thereof, and at least one additional thermistor on the second side protruding out of the finger probe device and configured to measure ambient temperature. This enables normalizing the finger temperature measured to the ambient temperature and thus to increase the reliability of the skin temperature measurement.

According to some embodiments, the accelerometer may be configured to provide measurements which may be directly associated with pain, such as shiver, spasm, etc. Additionally, or alternatively, the accelerometer may be configured to determine movement of the patient, which, for example, may cause changes in skin temperature and/or galvanic resistance and/or blood perfusion and thus enabling normalizing these signals to the subject's activity level.

According to some embodiments, the housing may include an additional PPG sensor preferably positioned so as to provide PPG measurements from a proximal end of the subject's finger. The combination of a proximal and a distal PPG sensor enables extraction of pulse speed wave for determining Pulse Transit Time (PTT) and blood pressure related parameters.

According to some embodiments, the housing further includes a cover, at least part of which may be made of a transparent plastic and configured to protect the PCB from direct contact as well as from penetration of liquids.

According to some embodiments, the housing may include a USB connector configured to allow connection of a USB cable, for example, for the purpose of charging the device.

According to some embodiments, the housing may include an additional connector configured to allow (electrical) connection of an electrode array thereto, as further described hereinbelow.

According to some embodiments, the housing may further include an attachment element configured to allow attachment of the strap of the body portion, thereto, as further described hereinbelow.

According to some embodiments, the housing may further include one or more transparent, protruding windows configured to accommodate indicator lamps e.g. lamps indicating operation and/or battery status.

According to some embodiments, the body portion is configured to be surroundingly positioned around the housing and is made of a semi-rigid material, such as, for example, silicon having a relatively high shore. According to some embodiments, the semi-rigid material has a type A shore of 30-55. According to some embodiments, the body portion includes a longitudinal aperture at its rear side through which the housing may be inserted, optionally in a reversible manner. According to some embodiments, once inserted, the part of the housing including its on/off button (and optionally also a company logo) is exposed through the aperture. Once surrounding the housing, the finger probe assumes a firm, but comfortable, scaffold for receiving and/or holding a subject's finger.

According to some embodiments, the body portion includes a longitudinal groove at its front side, the groove configured to accommodate the subject's finger. The groove ensures firm, correct and comfortable positioning of the subject's finger on the device. In addition, the groove advantageously prevents external light from reaching the PPG sensor and thus from interfering with PPG measurements, as further described hereinbelow.

According to some embodiments, the body portion includes a flexible strap or band configured to secure the finger on the device while minimally affecting blood perfusion. According to some embodiments, the strap may be made of the same material as the remainder of the body portion. Alternatively, the strap may be made from a material having a lower shore (e.g. shore A 20-40) and thus have increased flexibility as compared to the remainder of the body portion. According to some embodiments, the strap may include a plurality of apertures configured to prevent the finger from being warmed due to the wearing of the device. This advantageously increases patient comfort as well as ensures the reliability of galvanic skin resistance (GSR) measurements, as further elaborated herein. According to some embodiments, the strap further includes a plurality of bulges configured to prevent the finger from sliding relative to the device once secured by the strap yet without putting pressure on the finger which may result in perfusion problems.

According to some embodiments, the distal front end of the body portion may include a hood configured to cover the distal end of the subject's finger once secured to the device. According to some embodiments, the hood may be made of the same material (e.g. silicon) as the body portion and/or as the strap. According to some embodiments, the hood may have a shore lower than that of the body portion, but higher than that of the strap, e.g. a shore A 22. According to some embodiments, the shore of the hood material is configured to ensure tight grasping around the subject's finger while avoiding impairment of blood perfusion. According to some embodiments, the hood is configured to prevent penetration of external light. According to some embodiments, the hood may include an internal longitudinal groove, analogous to the longitudinal groove of the body portion. According to some embodiments, the longitudinal grooves, together, generate a tunnel-like compartment providing firm, correct and comfortable positioning of the subject's finger in the device, while preventing blood perfusion impairment. In addition, the tunnel-like compartment advantageously prevents external light from reaching the PPG sensor and thus from interfering with PPG measurements, as further described hereinbelow.

According to some embodiments, the elasticity of the hood material ensures that the tunnel allows for accommodation of fingers of different sizes. According to some embodiments, the hood may include a picture of the subject's nail so as to direct the subject to correct positioning of his finger within the finger probe. According to some embodiments, the finger probe may be suitable for essentially all finger sizes. According to some embodiments, the finger probe may be made of different sizes so as to accommodate different sized fingers. Non-limiting examples of suitable size classes include large (e.g. suitable for use with large adult fingers), medium (e.g. suitable for use in regular sized adult fingers), small (e.g. suitable for small adult and children fingers) and petite (e.g. suitable for use in infants and small children).

According to some embodiments, within the hood there is a stopper configured to prevent forward movement of the finger and to ensure its correct positioning relative to the sensors. According to some embodiments, the stopper may be of a height leaving a gap configured to accommodate long finger nails and to allow ventilation of the finger.

According to some embodiments, the body portion includes a PPG aperture configured to accommodate the PPG sensor (positioned and/or attached to the finger probe housing) in a liquid tight manner. According to some embodiments, the PPG sensor is accommodated within the PPG aperture so that light transmitted by the sensor reaches the subject's finger, when in use. It is understood that the sensor needs to be close to the subject's finger, but does not require direct contact therewith. According to some embodiments, the PPG aperture includes a transparent cover allowing the transmitted (and reflected) light to pass there through, yet preventing penetration of liquids into the housing.

According to some embodiments, the body portion may further include a thermistor aperture configured to accommodate a thermistor (or other skin temperature sensor positioned and/or attached to the finger probe housing) in a liquid tight manner. According to some embodiments, the thermistor aperture may be at least partially covered by a cover/seal configured to prevent liquids from penetrating the housing. According to some embodiments, the thermistor aperture and/or the cover may be sized and shaped to allow part of the thermistor, which require contact with the subject's finger, to penetrate into the compartment of the body portion formed by the hood.

According to some embodiments, the body portion may further include a USB aperture configured to allow a USB cable to be connected to a USB connector of the housing.

According to some embodiments, the body portion may further include an additional aperture allowing the connector of an electrode array to be connected to a matching connector within the housing.

According to some embodiments, the body portion may further include a strap attachment aperture configured to allow the attachment element of the housing to pass therethrough, as further described hereinbelow.

According to some embodiments, the body portion further includes one or more indicator apertures configured to receive the protruding windows of the housing, thereby allowing visibility of the indicator lamps once the housing is accommodated within the body portion.

According to some embodiments, the body portion further includes slits on each of it sides. The slits are sized and shaped to receive the wings of an electrode array, as further described hereinbelow.

According to some embodiments, there is provided a GSR electrode array configured for use with the herein described finger probe. According to some embodiments, the electrode array may be disposable. According to some embodiments, the electrode array may be a single use element. According to some embodiments, the GSR electrode array may be used more than once prior to disposal. According to some embodiments, the array may constitute an electrical circuit.

According to some embodiments, the electrode array includes an interphase portion forming two or more spaced apart compartments (also referred to as contact points) through which the electrodes are exposed for contact with the subject's finger. According to some embodiments, the compartments may include a conducting gel (or other conductive substance) capable of creating and/or enhancing the galvanic contact between the subject's finger and the electrodes. According to some embodiments, the interphase, and/or the conductive portion, may be flexible so as to ensure firm and comfortable contact with the subject's finger, thus enabling reliable measurements of the subject's galvanic skin response. According to some embodiments, the interphase portion may include an illustrative drawing or other element configured to guide the subject to correctly place his/her finger on the array.

According to some embodiments, the proximal end of the electrode array may include an electrical connector configured to connect the array to the PCB of the housing. According to some embodiments, the connector may be formed integrally with the array. According to some embodiments, the electrode array may include a bendable (conductive) portion interconnecting the interphase portion and the connector portion of the array.

According to some embodiments, the electrode array may include wings at each side thereof. The wings may be sized and shaped to be received within the slits of the finger probe's body portion, thereby stabilizing the electrode array relative to the finger probe. According to some embodiments, the connector and/or the wings of the electrode array may be of lower flexibility than the scaffold portion so as enable the connection of the connector and/or the sliding of the wings without causing them to be bent.

According to some embodiments, the slits may include a contact button positioned, such that sliding in the wings of the electrode into the slits presses upon the contact button, sending a signal to the PCB that an electrode array has been attached to the finger probe.

According to some embodiments, the interphase portion of the array may include an adhesive on the side thereof configured for facing the subject's finger. The adhesive is configured to ensure minimal movement of the finger relative to the array once correctly positioned. According to some embodiments, the adhesive may be covered by a removable cover configured to be removed prior to use. According to some embodiments, the cover may be shaped so as to enable its removal after the wings of the array have been slid into the slits of the finger probe.

According to some embodiments, the electrode array may include a piezoelectric sensor. Advantageously, the piezoelectric sensor may be arranged so as to enable determination of whether the finger attached to the array is kept straight, as a straight finger is important to the quality of the GSR measurements. According to some embodiments, more than one piezoelectric sensor may be included. Incorporation of two or more piezoelectric sensors may enable the extraction of pulse transient time (Ptt) readings. Additionally or alternatively, the Ptt readings may be extracted from signals obtained from a conjunction of a piezoelectric sensor and a PPG sensor. Additionally or alternatively, the Ptt readings may be extracted from signals obtained from two or more spaced apart PPG sensors, as described herein. The PPG sensor(s) and/or piezoelectric sensor(s) may be positioned such that the signals obtained are from a same arteriole, i.e. at the bottom of the finger and at the tip of the finger. According to some embodiments, the one or more piezoelectric sensors may, additionally or alternatively, be position also on the finger probe body and/or housing.

According to some embodiments, the PCB inside the housing may further include a microphone configured for voice recording. According to some embodiments, the microphone may be configured to record vocal complaints (such as, but not limited to, cries, whines, sighs of either elderly, pediatric, infant, newborns or pre-term subjects). The vocal complaints may be integrated with the signals obtained from the PPG sensor, the thermistor, the electrode array and optionally the accelerometer for monitoring and/or determining a subject's pain level.

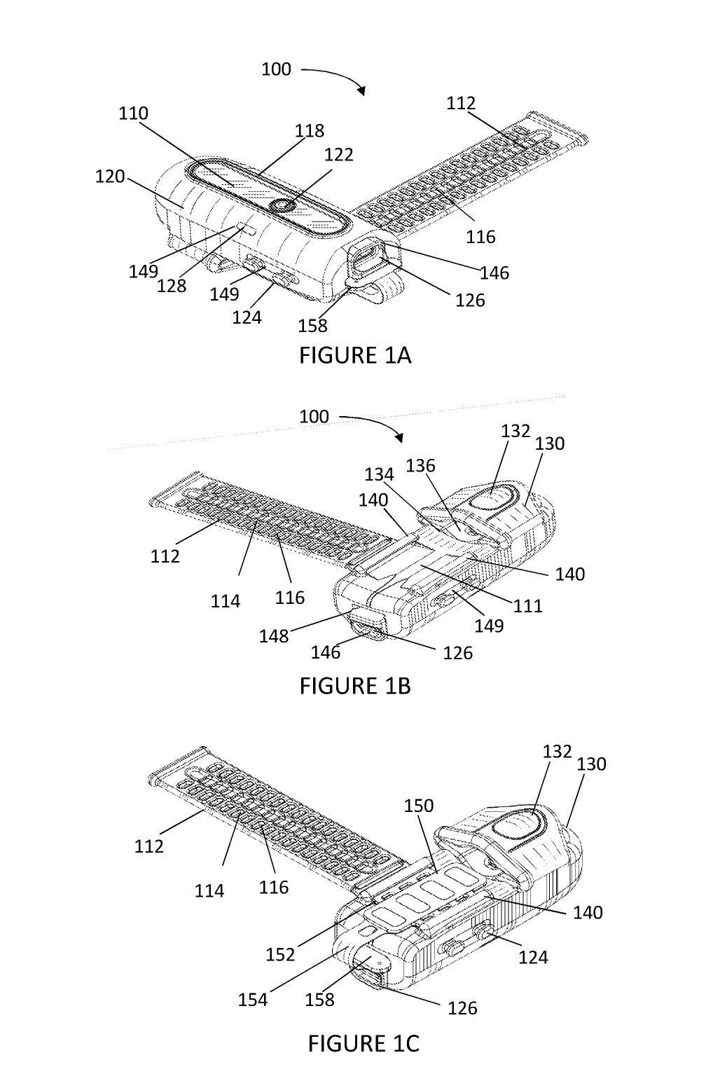

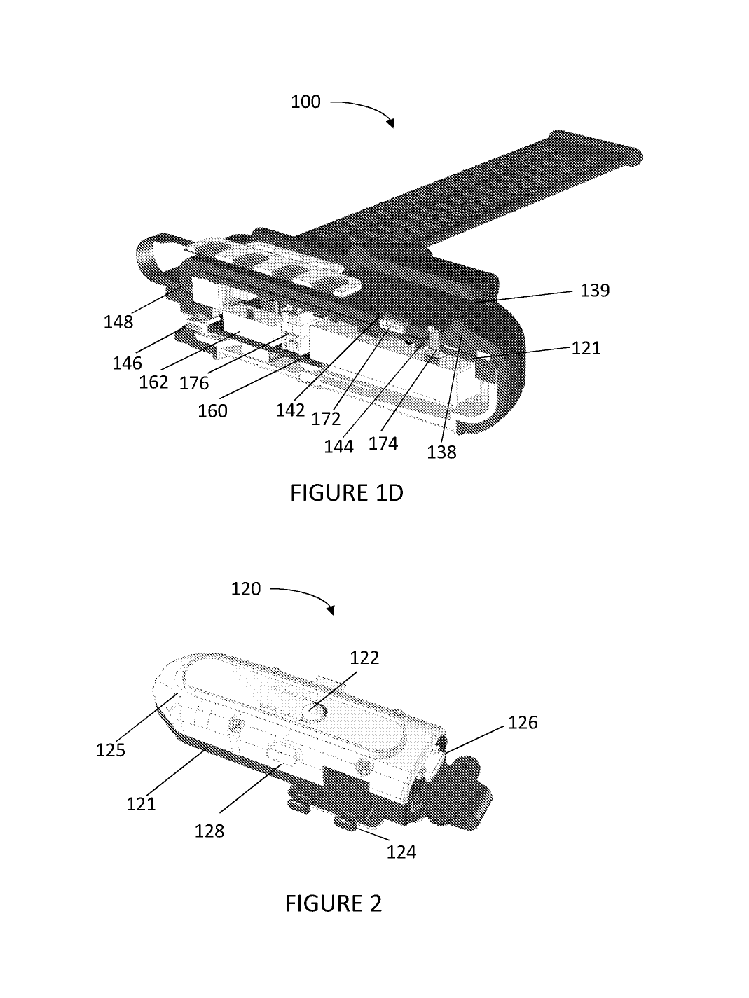

Reference is now made to the figures, which show views of a finger probe device 100 for monitoring pain, having a body portion 110 and a housing 120, according to some embodiments, as best seen in FIG. 1A and FIG. 1B.

Housing 120 (separately depicted in FIG. 2), is made of plastic and includes a scaffold 121 and a cover 125 made of a transparent plastic. On scaffold 121 is mounted a Printed Circuit Board (PCB) 160 with a PPG sensor 172, a thermistor 174 and an accelerometer 176, as depicted in FIG. 1D. PCB 160 includes a system on chip electrically that includes the PPG sensor 172, thermistor 174 and accelerometer 176 and includes an A2D and MCU that controls the chips with firmware that can be written on. PCB 160 has wireless capabilities (BLE) a rechargeable (optionally non-replaceable) battery 162, and a wire connector (USB) 126, for example, for the purpose of charging of the device.

Housing 120 further includes an attachment element 124 configured to allow attachment of strap 112 of body portion 110, thereto after being wrapped around a subject's finger (not shown).

Housing 120 also includes two transparent, protruding windows 128 configured to accommodate indicator lamps (not shown) such as lamps indicating operation and/or battery status of the device.

Body portion 110 is configured to be positioned around housing 120 and is made of a semi-rigid material, such as, for example, silicon having a relatively high shore. ensuring liquid tight interaction with housing 120. Body portion 110 includes a longitudinal aperture 118 at its rear side through which housing 120 is inserted. Once inserted, the part of housing 120 including its on/off button 122 is exposed through aperture 118.

Body portion 110 is shaped to form a longitudinal groove 111 at its front side (as depicted in FIG. 1B). Longitudinal groove 111 is configured to ensure firm and comfortable positioning of the subject's finger to the device.

Body portion 110 includes a flexible strap 112 configured to secure the finger on the device while minimally affecting blood perfusion. Strap 112 is made from a material having a lower shore than the remainder of body portion 110 and thus has increased flexibility enabling it to be bent around the subject's finger upon positioning without causing unnecessary pressure.

Strap 112 includes a plurality of apertures 116 configured to allow ambient air to reach the subject's finger and to prevent it from being warmed during use. This advantageously increases patient comfort as well as ensures the reliability of galvanic skin resistance (GSR) measurements, as changes in galvanic skin resistance due to warming of the finger are prevented or at least significantly reduced.

Strap 112 further includes a plurality of bulges 114 configured to prevent the finger from sliding relative to the device once secured by the strap without putting pressure on the finger.

At a distal front end of body portion 110 is a hood 130 configured to cover the distal end of the subject's finger once secured to the device, thereby shielding it from external light, yet without impairing blood perfusion. Hood 130 is shaped to form an internal longitudinal groove 134, analogous to longitudinal groove 111. Longitudinal grooves 111 and 134, together, form a tunnel-like compartment 136 providing comfortable positioning of the subject's finger in the device, while preventing blood perfusion impairment. Tunnel-like compartment 136 also prevents external light from reaching PPG sensor 172 and thus from interfering with PPG measurements. Optionally, hood 130 may include an illustration of a finger 132 guiding the subject to position his/her finger correctly, as shown in FIG. 1C and FIG. 1D.

Positioned within hood 130 is a stopper 138 (visible in FIG. 1D). Stopper 138 is configured to prevent forward movement of the finger and to ensure its correct positioning relative to PPG sensor 172 and thermistor 174. Between stopper 138 and hood 130 is a gap 139 configured to accommodate long finger nails as well as to allow ventilation of the finger.

Body portion 110 includes a PPG aperture 142 configured to accommodate PPG sensor 172 in a liquid tight manner, as seen in FIG. 1D. According to some embodiments, PPG aperture 142 includes a transparent cover (not shown) allowing transmitted (and reflected) light to pass therethrough, yet preventing penetration of liquids into housing 120.

Body portion 110 further includes a thermistor aperture 144 configured to accommodate thermistor 174 in a liquid tight manner.

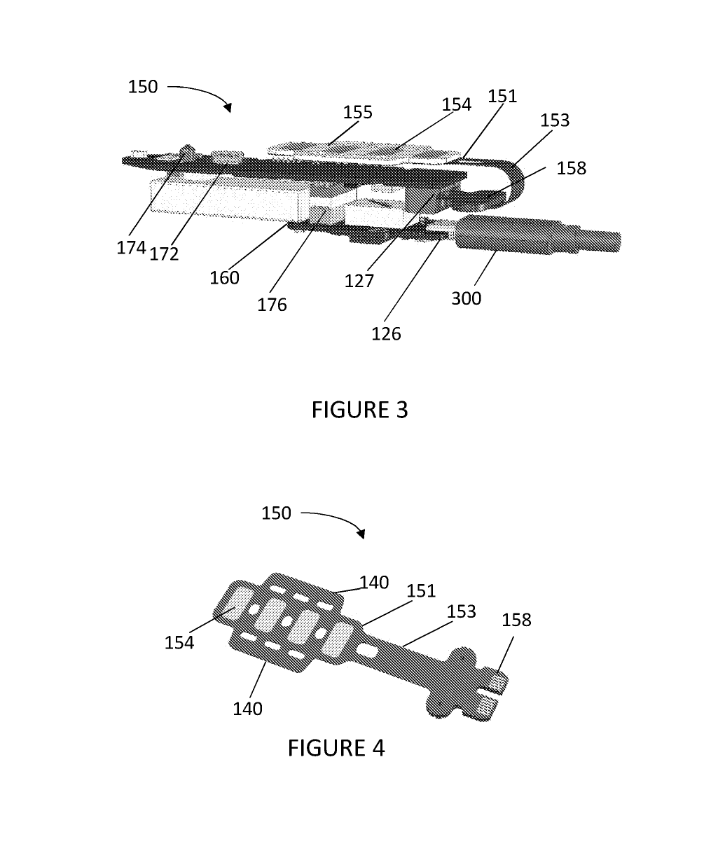

Body portion 110 also includes a USB aperture 146 configured to allow a USB cable (such as USB cable 300 shown in FIG. 3) to be connected to USB connector 126 of housing 120.

Body portion 110 further includes an additional aperture 148 allowing the connector 158 of an electrode array 150 to be connected to a matching connector 127 within the housing.

Body portion 110 also includes a strap attachment aperture 149 configured to allow attachment element 124 of housing 120 to pass therethrough.

Body portion 110 likewise includes two indicator apertures configured to receive the protruding windows of the housing, thereby allowing visibility of the indicator lamps once the housing is accommodated within body portion 110.

On its sides, body portion 110 further includes slits 140. Slits 140 are sized and shaped to receive wings 152 of electrode array 150.

Electrode array 150 is preferably provided as a separate disposable element configured for attachment to and/or use with finger probe device 100.

Electrode array 150 includes an electrically conductive flexible portion 151 (best seen in FIG. 4) serving as a scaffold for interphase portion 155 (best seen in FIG. 3). Interphase portion 155 is shaped to form compartments 154 (also referred to herein as contact points—here 4 contact points) exposing the electrodes for contact with the subject's finger. Compartments 154 also include a conductive biocompatible gel capable of creating and/or enhancing the galvanic contact between the finger and the electrodes. Interphase portion 155 may include an adhesive on the side thereof facing the subject's finger, when in use. The adhesive is configured to ensure minimal movement of the finger relative to the array once correctly positioned. According to some embodiments, the adhesive may be covered by a removable cover (not shown) configured to be removed prior to use.

The proximal end of electrode array 150 includes an electrical connector 158 configured to connect the array to PCB 160 of housing 120. A bendable (and optionally also conductive) portion 153 interconnects connector 158 and conductive portion 151.

Electrode array 150 further includes wings 152 at each side thereof. Wings 152 are sized and shaped to be received within slits 140 of body portion 110, thereby stabilizing electrode array 150 on finger probe device 100.

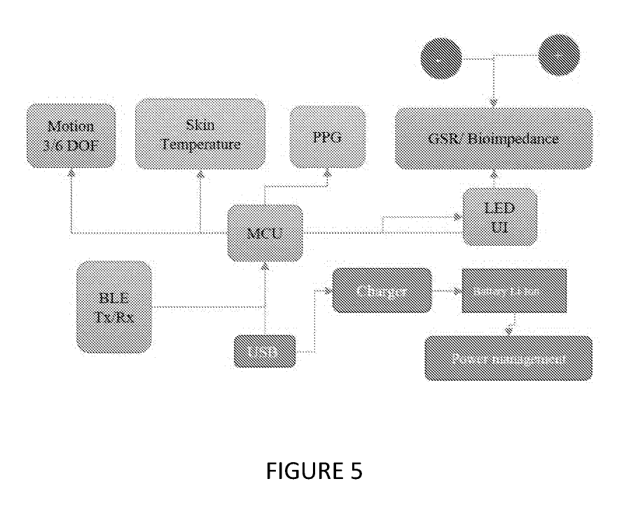

A block diagram of the pain monitoring system, is depicted in FIG. 5.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising”, when used in this specification, specify the presence of stated features, integers, steps, operations, elements, or components, but do not preclude or rule out the presence or addition of one or more other features, integers, steps, operations, elements, components, or groups thereof. Unless otherwise defined, all technical and scientific terms used herein have the same meaning as commonly understood by one of ordinary skill in the art to which this invention belongs.

Unless specifically stated otherwise, as apparent from the following discussions, it is appreciated that throughout the specification discussions utilizing terms such as “processing”, “computing”, “calculating”, “determining”, “estimating”, or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Embodiments of the present invention may include apparatuses for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

The invention may be described in the general context of computer-executable instructions, such as program modules, being executed by a computer. Generally, program modules include routines, programs, objects, components, data structures, and so forth, which perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1.-22. (canceled)

23. A finger probe for monitoring pain of a subject, the finger probe comprising a housing and a body portion configured to hold therein the housing,
wherein the body portion comprises a strap configured to secure the finger probe to the subject's finger, a first longitudinal groove configured to accommodate the subject's finger, and a hood configured to cover the distal end of the subject's finger from an opposite side thereof; and
wherein said housing comprises a Printed Circuit Board (PCB), a PPG sensor and at least one additional sensor.

24. The finger probe of claim 23, wherein said strap comprises a plurality of apertures configured to prevent heating of the subject's finger, when attached.

25. The finger probe of claim 23, wherein said strap comprises a plurality of bulges configured to generate friction and prevent movement of the subject's finger once secured.

26. The finger probe of claim 23, wherein said body portion is made of a material having a first shore and said strap is made of a material having a second shore, and wherein the second shore is lower than the first shore.

27. The finger probe of claim 23, further comprising two lateral slits configured to slidingly receive and secure wings of an electrode array.

28. The finger probe of claim 23, wherein said body portion comprises a longitudinal aperture exposing at least an on/off button of said housing.

29. The finger probe of claim 23, wherein the hood comprises a second longitudinal groove, wherein said first and second longitudinal grooves form a tunnel-like compartment configured to accommodate the subject's finger and to prevent penetration of ambient light.

30. The finger probe of claim 23, wherein said PPG sensor is positioned at a distal end of said housing underneath said tunnel-like compartment.

31. The finger probe of claim 31, wherein said body portion comprises a PPG aperture within said tunnel-like compartment and above said PPG sensor, said PPG aperture configured to allow light transmitted by said PPG sensor to reach the subject's finger.

32. The finger probe of claim 32, wherein said PPG aperture is covered by a transparent cover allowing light transmitted by said PPG sensor to pass there through, while preventing penetration of liquids into the housing.

33. The finger probe of claim 23, wherein said at least one additional sensor comprises a thermistor configured to monitor skin temperature of the subject's finger.

34. The finger probe of claim 33, wherein said thermistor is positioned at a distal end of said housing underneath said tunnel like compartment.

35. The finger probe of claim 34, wherein said body portion further comprises a thermistor aperture configured to allow contact between said thermistor and the subject's finger.

36. The finger probe of claim 23, wherein said body portion further comprises a stopper positioned within said tunnel-like compartment, said stopper configured to prevent forward movement of the subject's finger.

37. The finger probe of claim 23, wherein said at least one additional sensor further comprises an accelerometer.

38. The finger probe of claim 23, wherein said PCB is electrically connected to said PPG sensor, to said at least one additional sensor and to a rechargeable battery, and wherein said PCB is configured for wireless communication with a remote monitor and/or computer.

39. A pain monitoring system comprising:
a finger probe comprising a housing and a body portion configured to hold therein the housing, wherein the body portion comprises a strap configured to secure the finger probe to a subject's finger, a first longitudinal groove configured to accommodate the subject's finger, and a hood configured to cover the distal end of the subject's finger from an opposite side thereof; and wherein said housing comprises a Printed Circuit Board (PCB), a PPG sensor and at least one additional sensor; and
an electrode array configured to monitor galvanic skin resistance (GSR) on the subject's finger, the electrode array comprising an electrically conductive flexible portion comprising GSR electrodes and an interphase portion, wherein the interphase portion is shaped to form at least two compartments configured to enable contact between the subject's finger and the GSR electrodes, wherein the electrode array comprises lateral wings configured to secure said electrode array to a finger probe; and wherein a proximal end of said electrically conductive flexible portion comprises an electrical connector configured to electrically connect the electrode array to the PCB of the finger probe.

40. The pain monitoring system of claim 39, wherein the at least two compartments comprise a conductive biocompatible gel capable of creating and/or enhancing galvanic contact between the subject's finger and the GSR electrodes.

41. The pain monitoring system of claim 39, wherein the interphase portion comprises an adhesive on the side thereof configured to face the subject's finger.

42. The pain monitoring system of claim 39, wherein the adhesive is covered by a removable cover configured to be removed prior to use.
